(19) United States
(12) Patent Application Publication
(10) Pub. No.: US 2009/0004742 A1
(43) Pub. Date: Jan. 1, 2009

(54) SELECTION OF ANTIGEN-SPECIFIC T CELLS

(75) Inventors: Duane A. Mitchell, Durham, NC (US); John Sampson, Durham, NC (US)

Correspondence Address: BANNER & WITCOFF, LTD., 1100 13th STREET, N.W., SUITE 1200, WASHINGTON, DC, 20005-4051, US

(73) Assignee: Duke University, Durham, NC (US)

(21) Appl. No.: 12/144,318

(22) Filed: Jun. 23, 2008

Related U.S. Application Data
(63) Continuation of application No. 11/931,122, filed on Oct. 31, 2007, now abandoned.
(60) Provisional application No. 60/863,916, filed on Nov. 1, 2006.

(51) Int. Cl. C12N 15/87; C12N 5/08

(52) U.S. Cl. 435/455; 435/372.3

(57) ABSTRACT

The requirement of T cell activation for efficient expression of genes after messenger ribonucleic acid (mRNA) transfection is leveraged to identify and enrich antigen-specific T cells responding to antigen-pulsed dendritic cells (DCs). RNA transfection of marker genes is used for the selection and enrichment of antigen-specific T cells for use in adoptive immunotherapy. RNA-modified T cells are also used for the generation of enhanced effector populations for use in adoptive immunotherapy. Genes whose transient expression may significantly enhance the in vivo function of T cells (i.e., migratory receptors, anti-apoptotic genes or cytokines enhancing T cell proliferation/differentiation) are used in this modality.

### TECHNICAL FIELD OF THE INVENTION

This invention is related to the area of adoptive immunotherapy. In particular, it relates to generation of populations of antigen specific T cells useful for adoptive immunotherapy.

### BACKGROUND OF THE INVENTION

Despite remarkable advancements in imaging modalities and treatment options available to patients diagnosed with malignant brain tumors, the prognosis for those with high-grade lesions remains poor. The imprecise mechanisms of currently available treatments to manage these tumors do not spare damage to the normal surrounding brain and often result in major cognitive and motor deficits. Immunotherapy holds the promise of offering a potent, yet targeted, treatment to patients with brain tumors, with the potential to eradicate the malignant tumor cells without damaging normal tissues. The T cells of the immune system are uniquely capable of recognizing the altered protein expression patterns within tumor cells and mediating their destruction through a variety of effector mechanisms. Adoptive T-cell therapy is an attempt to harness and amplify the tumor-eradicating capacity of a patient's own T cells and then return these effectors to the patient in such a state that they effectively eliminate residual tumor. Although this approach is not new to the field of tumor immunology, new advancements in our understanding of T-cell activation and function and breakthroughs in tumor antigen discovery hold great promise for the translation of this modality into a clinical success.

There is a continuing need in the art to improve the methods of collecting T-cells useful for adoptive therapies.

### SUMMARY OF THE INVENTION

According to one embodiment a method is provided for marking and separating antigen-specific, activated T cells in a population. A first population of T cells is transfected with mRNA encoding a detectable marker. The first population of T cells is withdrawn from a patient and comprises one or more activated, antigen-specific T cells. T cells which express the marker are separated from those which do not express the marker to form a second and third population of T cells. The second population of T cells comprises T cells which express the marker and the second population is enriched for antigen-specific, activated T cells relative to the first population. The third population of T cells comprises T cells which do not express the marker and the third population is depleted for antigen-specific, activated T cells relative to the first population.

Another embodiment of the invention is an isolated population of T cells which comprises T cells which express a marker encoded by a transfected exogenous mRNA. The T cells are specifically activated by an antigen. The population comprises at least 95% activated T cells which are specific for the antigen.

Yet another embodiment of the invention is an isolated population of T cells which comprises T cells which are not specifically activated by an antigen. The population comprises less than 5% activated T cells which are specific for the antigen.

Still another embodiment of the invention provides a method of selectively modifying biological function of antigen-specific, activated T cells in a population. A first population of T cells is transfected with mRNA encoding a biologically active protein. The first population of T cells is withdrawn from a patient and comprises one or more activated, antigen-specific T cells as well as non-activated T cells. The activated, antigen-specific T cells are selectively transfected and selectively modified by expressing the biologically active protein.

According to another embodiment a population of T cells withdrawn from a patient comprises one or more activated, antigen-specific T cells. The activated antigen-specific T cells are transiently transfected with an mRNA encoding a biologically active protein. The activated antigen-specific T cells express said biologically active protein thereby altering cellular behavior.

These and other embodiments which will be apparent to those of skill in the art upon reading the specification provide the art with new reagents and methods for manipulating and using T cells.

### BRIEF DESCRIPTION OF THE DRAWINGS

FIG. 1. High efficiency gene expression inhuman T cells using RNA electroporation. Polyclonal stimulation (anti-CD3) results in efficient RNA transfection of human T cells.

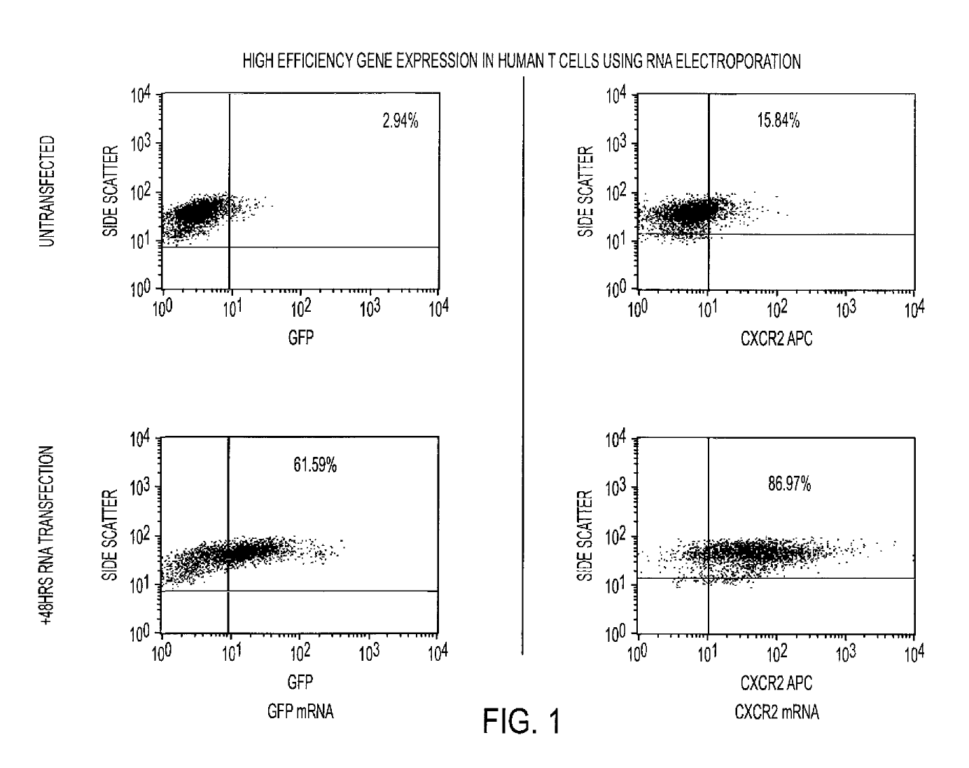

FIG. 2. Selection of Antigen-Specific T cells Using RNA Transfection. T cells stimulated with antigen-presenting cells pulsed with CMV peptide (pp65). Top panel shows 24.5% of all CD8+ T cells express GFP after transfection of RNA. Bottom panel demonstrates that GFP expression occurs exclusively within the pp 65 tetramer positive T cells but not the tetramer negative T cells. Tetramer identifies specific T cells (tet+) from non-specific T cells (tet−).

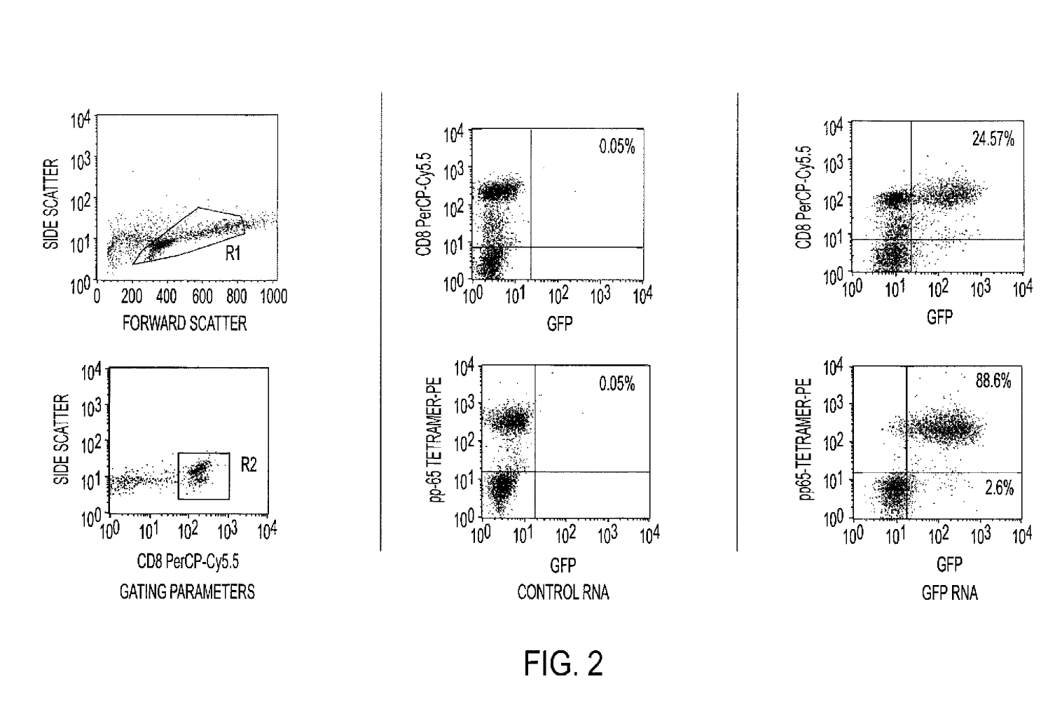

FIG. 3. Expansion of sorted GFP RNA transfected T cells. T cells stimulated against whole pp 65 antigen were transfected with GFP RNA, sorted as GFP+ and GFP− cells and expanded further in culture using high dose IL-2 (100 U/ml). Results show that only cells identified by RNA transfection of GFP were capable of further expansion demonstrating that RNA transfection separates cells capable of being expanded for use in adoptive immunotherapy from non-responding populations of T cells.

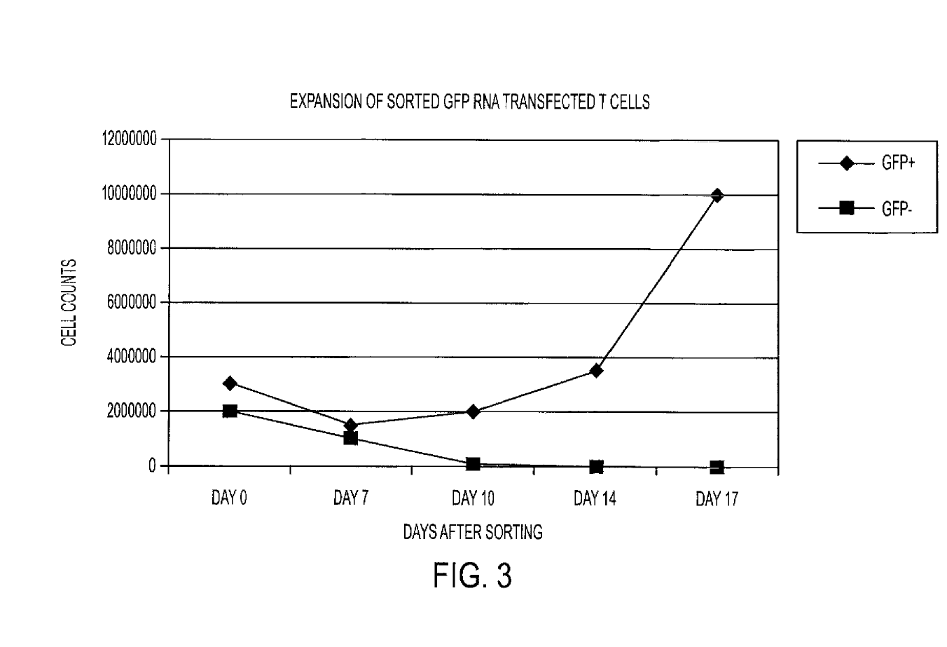

FIG. 4. Expression of CXCR2 Chemokine Receptor in Antigen-Specific T cells. We demonstrate that the expression of RNA in antigen specific T cells can be used to selectively modify specific populations of T cells within bulk cultures to provide enhanced function or regulation of these cells. This can be utilized to enhance the chemotactic function of these cells (shown below), selectively kill or provide resistance to antigen specific T cells, or provide any number of specific functions to only those cells of interest while leaving non-responding T cells unmodified.

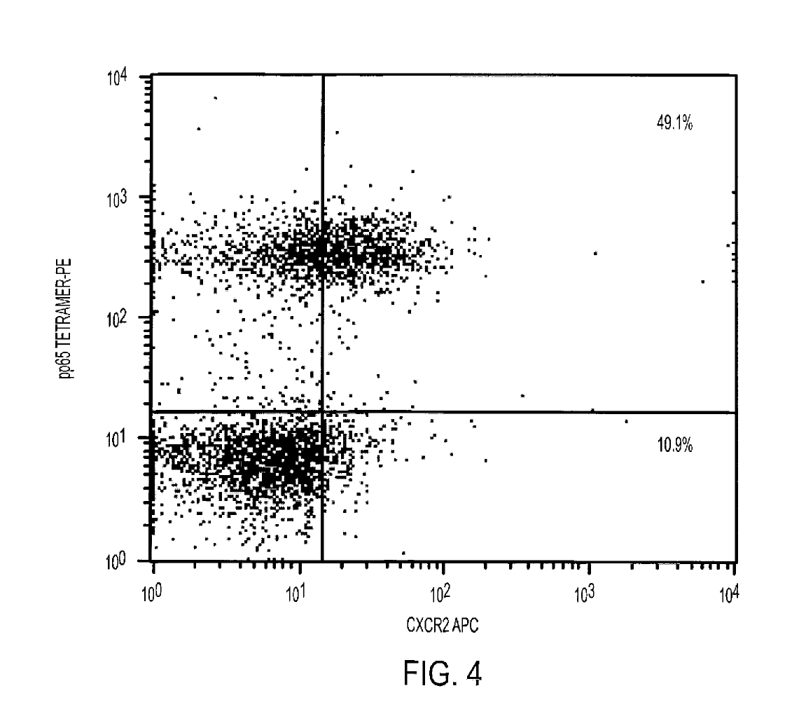

FIG. 5. In vitro chemotaxis of CXCR2 and GFP RNA transfected T cells toward IL-8. Enhanced chemotaxis in CXCR2 modified T cells toward IL-8.

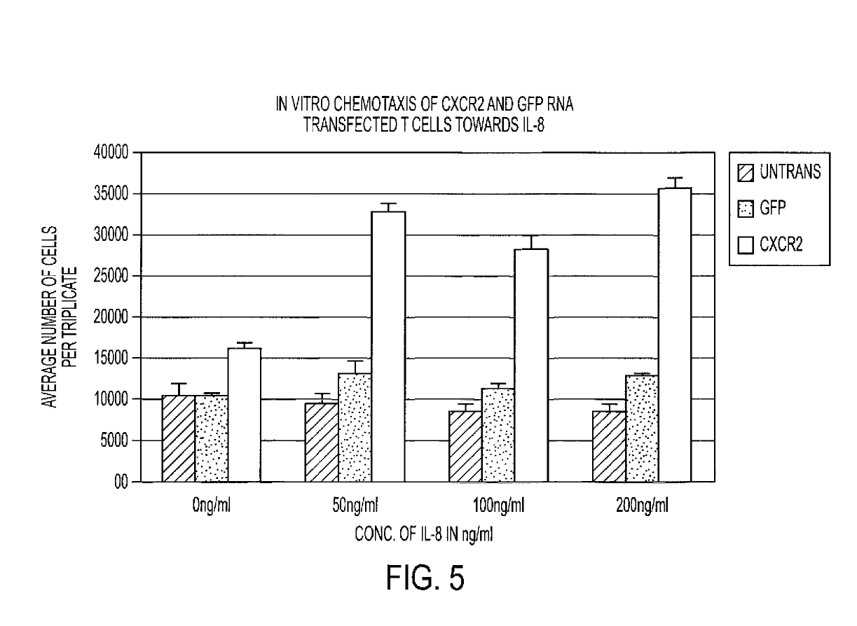

### DETAILED DESCRIPTION OF THE INVENTION

The inventors have developed a method for isolating antigen-specific T cells in a highly efficient way so that populations useful for adoptive immunotherapy can be generated. Beneficially, the isolation permits the depletion of T_{reg} cells from the antigen-specific T cells.

Transfection with mRNA can be accomplished by any means known in the art. According to one method, electroporation is used to facilitate transfection. Other chemical and physical treatments for enhancing transfection by mRNA can be used, including but not limited to thermal shock, adjustment of salts, such as calcium, liposomes, and other permeabilizing treatments.

T cells for transfection can be obtained by any method known in the art. They can be obtained from the same patient to whom they will be administered after transfection. They can be obtained from other allogeneic persons, such as brothers, sisters, parents, offspring, and unrelated persons. Typically T cells are obtained from peripheral blood lymphocytes or from lymph nodes, or from tumor infiltrating lymphocytes. They can be separated from other cells in the blood by density gradient centrifugation, for example. T cells can be isolated using antibodies for specific antigens found on T cells. Such antigens and antibodies are known in the art and can be used as desired.

Preparations of mRNA encoding a detectable marker can be prepared by any technique known in the art. Detectable markers can be anything which is convenient for detection and not considered harmful to the T cells or the patient. Marker proteins may be enzymes which are detectable using a chromogenic substrate, for example. Alternatively, marker proteins can be luminescent or fluorescent. In other embodiments the marker can be any protein which is detectable using an antibody specific for the marker.

Activated, antigen-specific T cells in a population of T cells can be those which are initially present on withdrawal of the T cells from a patient. Alternatively, the T cells withdrawn can be stimulated with antigen in vitro. Antigens which can be used to stimulate the T cells include, without limitation, tumor-associated antigens, tumor-specific antigens, viral antigens, parasite antigens, allogeneic cells as an antigen, antigen obtained from allogeneic cells, antibodies against specific T cell receptors, and irradiated cancer cells withdrawn from the patient. The antigens can be presented to the T cells in any way known in the art, including on a dendritic cell which has been pulsed with the antigen, on a dendritic cell which has been transfected with a nucleic acid encoding the antigen.

Separation of T cells which express the marker from those which do not express the marker can be accomplished by any means known in the art. One method employs an immunological separation in which antibodies are used to select for cells which express the marker. Another method employs fluorescent activated cell sorting (FACS).

Upon separation, two populations are formed. One population predominantly expresses the marker and the other predominantly does not express the marker. When excellent separations are performed, at least 96, 97, 98, or 99 percent of the population of marker expressing cells are also activated T cells specific for the antigen. Thus undesired cells, such as T_{reg} can be depleted from the activated T cell population. When excellent separations are performed less than 4, 3, 2, or 1 percent of the cells in the marker non-expressing cells are activated T cells specific for the antigen. Populations which are predominantly activated T cells specific for a desired antigen are excellent for use in adoptive cell immunotherapy protocols. They can be administered to a patient according to any of the routine methods for infusion of T cells into the circulation. Populations which are predominantly non-activated T cells can be used, inter alia, for allotransplantation.

T cells stimulated against specific antigens (viral, tumor, allogeneic antigens, endothelial, etc) and transfected with RNA can be specifically modified thereby forming two populations of T cells. The capacity to modify T cells in an antigen-specific manner permits selective modification, killing, or separation of antigen specific T cells that can be administered for therapeutic use. The administration of T cell populations selectively modified using RNA transfer, with or without separation of the modified from the unmodified populations, permits several novel capacities to be conferred on specific T cell subpopulations including but not limited to altered trafficking in vivo, altered proliferative advantage or attenuation, altered differentiation, altered effector function, and altered survival due to apoptosis inhibition or induction. Specific mRNAs which can be used to achieve these capacities include, but are not limited to those encoding: CXCR2, CXCR4, receptors for MIP-1α and -1β, CCR7, CCR5, and NGF-R for trafficking; IL-7 and/or IL-7 receptor for differentiation; IL-2 and/or IL-2R, IL-15 and/or IL-15R for proliferative advantage; BCL-2, BCL-X, survivin, and Lung Kruppel-Like Factor for inhibition of apoptosis; FasL receptor, PDL-1, Pseudomonas toxin, and caspases for induction of apoptosis; FasL, granzyme B, TNF-α, IFN-γ, anti-VEGF antibodies for effector function. In addition, siRNA to any of IL-2R, IL-2, NFₖB, IL-15, and IL-15R can be used for attenuation of proliferation. As used herein, the term “mRNA” includes siRNA or miRNA. These proteins and mRNAs are all known in the art.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### EXAMPLE 1

### Selection of Antigen-Specific T Cells Using RNA Transfection of Marker Genes

The enrichment of antigen-specific T cells for use in adoptive immunotherapy is of considerable interest in order to increase the efficacy of the delivered population of cells. We hypothesized that the requirement of T cell activation for efficient expression of genes after messenger ribonucleic acid (mRNA) transfection could be leveraged to identify and enrich antigen-specific T cells responding to antigen-pulsed dendritic cells (DCs). We utilized mRNA encoding for green fluorescent protein (GFP) as a marker gene for evaluating the ability to target antigen-specific T cells using mRNA transfection.

Human T cells from HLA-A2+ donors were stimulated with autologous DCs pulsed with a CMV-specific, pp 65 peptide, or transfected with mRNA encoding for full-length pp 65. Stimulated T cells were electroporated with mRNA encoding for GFP and expression of GFP in antigen-specific and non-specific T cell populations was examined by tetramer analysis and cytokine flow cytometry.

In cultures stimulated by DCs pulsed with pp 65 peptide expression of GFP was observed in a high proportion of CMV-specific T cells (60-100%) with little to no expression in non-specific T cells (0-10%). Sorting of GFP(+) and GFP(−) T cells after stimulation with DCs presenting full-length pp 65 revealed that all of the antigen-specific T cells segregated with the GFP+ population and represented a 25 fold enrichment of antigen-specific T cells.

RNA transfection of marker genes represents a novel platform for the selection and enrichment of antigen-specific T cells for use in adoptive immunotherapy.

### EXAMPLE 2

### RNA-Modified T cells for Use in Adoptive Immunotherapy

We have examined messenger ribonucleic acid (mRNA) transfection as a novel platform for transiently modifying the function of T cells for use in adoptive immunotherapy. We evaluated the expression of the chemokine receptor, CXCR2, in activated T cells in its capacity to enhance migration of T cells toward chemokines produced by malignant gliomas such as IL-8 and GRO-α, and towards a human cytomegalovirus (HCMV) specific chemokine, UL146, which is secreted from CMV-infected cells.

cDNA for CXCR2 and green fluorescent protein (GFP) was cloned into a RNA-expression vector and mRNA synthesized using in vitro transcription. mRNA was introduced into activated human T cells (stimulated with anti-CD3 coated plates or antigen-pulsed dendritic cells) using electroporation. Expression of CXCR2 and/or GFP was examined using flow cytometry and chemotaxis toward CXCR2-specific ligands was measured using trans-well migration assays.

Expression of CXCR2 and GFP was observed in a high proportion of electroporated T cells (60-85%) with peak expression at 48 hrs post transfection and for duration of 5-7 days. CXCR2 transfected T cells exhibited enhanced migration toward IL-8, Gro-α, and UL146 compared to untransfected or GFP transfected T cells.

RNA-modified T cells represent a simple and novel platform for the generation of enhanced effector populations for use in adoptive immunotherapy. Genes whose transient expression may significantly enhance the in vivo function of T cells (i.e. migratory receptors, anti-apoptotic genes or cytokines enhancing T cell proliferation/differentiation) may have considerable potential for application in this modality.

1. A method of marking and separating antigen-specific, activated T cells in a population, comprising the steps of:
transfecting a first population of T cells with mRNA encoding a detectable marker, wherein the first population of T cells is withdrawn from a patient and comprises one or more activated, antigen-specific T cells;
separating T cells which express the marker from those which do not express the marker to form a second and third population of T cells, wherein the second population of T cells comprises T cells which express the marker and the second population is enriched for antigen-specific, activated T cells relative to the first population, wherein the third population of T cells comprises T cells which do not express the marker and the third population is depleted for antigen-specific, activated T cells relative to the first population.

2. The method of claim 1 wherein, prior to the step of transfecting, the first population of T cells is contacted with an antigen or an antibody specific for a receptor for the antigen, to increase number of activated T cells in the first population which are specific for the antigen.

3. The method of claim 1 wherein, subsequent to the step of separating, the second population is administered to the patient.

4. The method of claim 1 wherein the second population comprises at least 95% of the antigen specific activated T cells present in the first population.

5. The method of claim 1 wherein the second population comprises at least 96% of the antigen specific activated T cells present in the first population.

6. The method of claim 1 wherein the second population comprises at least 97% of the antigen specific activated T cells present in the first population.

7. The method of claim 2 wherein the antigen is a tumor-associated antigen.

8. The method of claim 2 wherein the antigen is a tumor-specific antigen.

9. The method of claim 2 wherein the antigen is a viral antigen.

10. The method of claim 2 wherein the antigen is a parasite antigen.

11. The method of claim 2 wherein the T cells are contacted with allogeneic cells as an antigen.

12. The method of claim 2 wherein the T cells are contacted with an antigen obtained from allogeneic cells.

13. The method of claim 2 wherein the antigen that is contacted with the first population of T cells is on a dendritic cell which has been pulsed with the antigen.

14. The method of claim 2 wherein the antigen that is contacted with the first population of T cells is on a dendritic cell which has been transfected with a nucleic acid encoding the antigen.

15. The method of claim 2 wherein the antigen that is contacted with the first population of T cells is on irradiated cancer cells withdrawn from the patient.

16. The method of claim 1 wherein the T cells are obtained from peripheral blood mononuclear cells.

17. The method of claim 1 wherein the T cells are obtained from lymph nodes.

18. The method of claim 1 wherein the T cells are obtained from tumor infiltrating lymphocytes.

19. The method of claim 1 wherein less than 3% of the third population is antigen-specific T cells.

20. An isolated population of T cells which comprises T cells which express a marker encoded by a transfected exogenous mRNA and which are specifically activated by an antigen, wherein the population comprises at least 95% activated T cells which are specific for the antigen.

21. The isolated population of claim 21 wherein the population comprises at least 96% activated T cells which are specific for the antigen.

22. The isolated population of claim 21 wherein the population comprises at least 97% activated T cells which are specific for the antigen.

23. An isolated population of T cells which comprises T cells which are not specifically activated by an antigen, wherein the population comprises less than 5% activated T cells which are specific for the antigen.

24. The isolated population of claim 24 wherein the population comprises less than 4% activated T cells which are specific for the antigen.

25. The isolated population of claim 24 wherein the population comprises less than 3% activated T cells which are specific for the antigen.

26. The isolated population of claim 24 which is made by the process of claim 1.

27. A method of selectively modifying biological function of antigen-specific, activated T cells in a population, comprising the steps of:
transfecting a first population of T cells with mRNA encoding a biologically active protein, wherein the first population of T cells is withdrawn from a patient and comprises one or more activated, antigen-specific T cells as well as non-activated T cells, whereby the activated, antigen-specific T cells are selectively transfected and selectively modified by expressing the biologically active protein.

28. The method of claim 28 further comprising the step of: separating T cells which express the biologically active protein from those which do not express the biologically active protein to form a second and third population of T cells, wherein the second population of T cells comprises T cells which express the biologically active protein and the second population is enriched for antigen-specific, activated T cells relative to the first population, wherein the third population of T cells do not express the biologically active protein and the second population is depleted for antigen-specific, activated T cells relative to the first population.

29. A population of T cells withdrawn from a patient and comprising one or more activated, antigen-specific T cells, wherein the activated antigen-specific T cells are transiently transfected with an mRNA encoding a biologically active protein whereby the activated antigen-specific T cells express said biologically active protein thereby altering cellular behavior.

30. The population of claim 29 wherein the biologically active protein is selected from the group consisting of CXCR2, CXCR4, receptors for MIP-1α and -1β, CCR7, CCR5, NGF-R, IL-7, IL-7 receptor, IL-2, IL-2R, IL-15, IL-15R, BCL-2, BCL-X, survivin, Lung Kruppel-Like Factor, FasL receptor, PDL-1, Pseudomonas toxin, caspases, FasL, granzyme B, TNF-α, IFN-γ, anti-VEGF antibodies, and combinations thereof.
